(19) United States
(12) Patent Application Publication
(10) Pub. No.: US 2019/0216627 A1
(43) Pub. Date: Jul. 18, 2019

(54) JOINT BRACE

(71) Applicant: Kurt Requa, Rochester, MN (US)

(72) Inventor: Kurt Requa, Rochester, MN (US)

(73) Assignee: GatesReed LLC, Rochester, MN (US)

(21) Appl. No.: 16/185,129

(22) Filed: Nov. 9, 2018

Related U.S. Application Data
(60) Provisional application No. 62/611,515, filed on Dec. 28, 2017.

(51) Int. Cl. A61F 5/01

(52) U.S. Cl. CPC A61F 5/01; A61F 2005/0179; A61F 5/0123

(57) ABSTRACT

A joint brace comprises a first frame adapted to be secured onto a body part on the first side of a joint, a second frame adapted to be secured onto a body part on the other side of a joint, a hinge mechanism connecting the first frame to the second frame so the first frame and the second frame can rotate relative to one another about a hinge axis that is adapted to be generally aligned with the axis of rotation of the joint, and a bias system that biases the first frame away from the second frame and towards an extended position. In one version, the bias is applied for more than about 30 degrees of flexion of the first frame relative to the second frame. In another version, is removable so that it can be selectively installed and removed by the user.

### PRIORITY

The present application claims the benefit of domestic priority based on U.S. Provisional Patent Application 62/611,515 filed on Dec. 28, 2017, the entirety of which is incorporated herein by reference.

### BACKGROUND

Surprisingly, support braces for athletes have only been in existence for a few decades. In the late 1960's, Joe Namath became the first professional athlete to wear a conventional knee brace to prevent lateral motion of his weak knee. As textiles, such as neoprene and polyesters became popular in the 1970's, football players began wearing more advanced, lightweight knee braces. As lightweight plastic compounds became widely available, kinesiologists began developing lightweight, customized braces for ankles and knees. While many of these developments have improved the designs of brace systems, existing braces do not offer the ability to tailor the amount of torsion applied to the knee or other joint and/or supported by the knee or other joint.

Therefore, there is a need for an improved joint brace. There is further a need for a joint brace with a spring that biases the joint brace toward a desired position. There is still further a need for a joint brace with a torsion spring that biases the joint brace toward an extended position. There is still further a need for a joint brace that can adjust the amount of torque applied to the joint. There is still further a need for a joint brace that can be retrofitted with a one or more springs.

### SUMMARY

The present invention satisfies these needs. In one aspect of the invention, a joint brace offers an improvement over existing joint braces and/or offers the ability to improve existing joint braces.

In another aspect of the invention, a joint brace includes a spring that biases the joint brace toward a desirable position.

In another aspect of the invention, a joint brace includes a torsion spring that biases the brace toward an extended position.

In another aspect of the invention, a joint brace can adjust the amount of torque applied to a joint.

In another aspect of the invention, an existing joint brace can be retrofitted to include a biasing system.

In another aspect of the invention, a joint brace comprises a first frame adapted to be secured onto a body part on the first side of a joint, a second frame adapted to be secured onto a body part on the other side of a joint, a hinge mechanism connecting the first frame to the second frame so the first frame and the second frame can rotate relative to one another about a hinge axis that is adapted to be generally aligned with the axis of rotation of the joint, and a bias system that biases the first frame away from the second frame and towards an extended position, wherein the bias is applied for more than about 30 degrees of flexion of the first frame relative to the second frame.

In another aspect of the invention, a joint brace comprises a first frame adapted to be secured onto a body part on the first side of a joint, a second frame adapted to be secured onto a body part on the other side of a joint, a hinge mechanism connecting the first frame to the second frame so the first frame and the second frame can rotate relative to one another about a hinge axis that is adapted to be generally aligned with the axis of rotation of the joint, and a bias system that biases the first frame away from the second frame and towards an extended position, wherein the bias system is removable so that it can be selectively installed and removed by a user.

In another aspect of the invention, a method of bracing a joint comprises securing a first frame to a body part on a first side of a joint; securing a second frame to a body part on the other side of the joint, the first and second frames being connected to one another by a hinge mechanism; attaching to at least the first frame a biasing system that biases the first frame toward an extended position; and removing the biasing system from the first frame when it is desired to remove the bias.

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1 is a schematic side view of a joint brace of the invention;

FIG. 2 is a schematic side view of another version of a joint brace of the invention;

FIG. 3 is a schematic side view of another version of a joint brace of the invention;

FIG. 4A is schematic side view of a torsion spring for use with the joint brace of FIG. 3;

FIG. 4B is a schematic side view of a joint brace of the invention installed with the torsion spring of FIG. 4B;

FIG. 5A is a schematic side view of a dual torsion spring for use with the joint brace of FIG. 3; and

FIG. 5B is a schematic side view of a joint brace of the invention installed with the dual torsion spring of FIG. 5A.

### DESCRIPTION

The present invention relates to a joint brace. In particular, the invention relates to a joint brace with a biasing system. Although the invention is illustrated and described in the context of being useful for biasing a flexion joint, the present invention can be used in other ways, as would be readily apparent to those of ordinary skill in the art. Accordingly, the present invention should not be limited just to the examples and embodiments described herein.

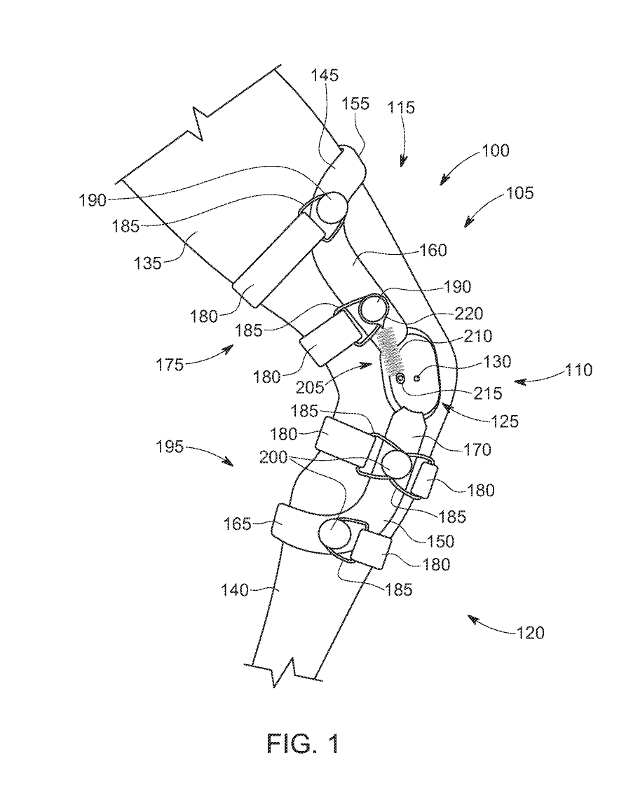

FIG. 1 shows a joint brace 100 according to a version of the present invention. In this version, the joint brace 100 is shown in the form of a knee brace 105 being used to support a knee joint 110. Alternatively, the joint brace 100 can be used to support another joint, such as an elbow, ankle, shoulder, wrist, or the like. The joint brace 100 can be used to help rehabilitate an injured joint and/or can be used to prevent injury to a joint during an activity such as an athletic activity. Though described in the context of a knee brace 105 herein, the joint brace 100 is applicable to at least the joints described above with minor modification. A joint brace 100, also known as a joint orthosis, is a specially designed external medical device constructed of lightweight and durable materials and designed to provide biomechanical assistance and support to a joint. In particular, a knee brace 105 or knee orthosis is designed to provide stability and support to the knee 110 and the surrounding ligaments, tendons, and muscles. The knee brace 105 can be used for guiding, limiting, immobilizing and/or controlling the knee joint; for providing assistance with general movement of the knee joint; for restricting movement of the knee joint in a given direction; for reducing the forces of weight-bearing on the knee joint; for enhancing rehabilitation from fractures of the knee joint; and/or for correcting the function or shape of the knee joint, specifically for facilitating movement and/or pain reduction.

The joint brace 100 includes a first frame 115 and a second frame 120 that are connected at a hinge mechanism 125. The first frame 115 is secured to the user on one side of the joint, and the second frame 120 is secured to the user on the other side of the joint so that the hinge mechanism is generally positioned in proximity to the joint and so that a hinge axis 130 is generally in line with a rotational axis of the joint. In the version, of FIG. 1 the joint brace 100 comprises a knee brace 105 where the first frame 115 is secured to the upper leg or thigh 135 of a user, the second frame 120 is secured to the lower leg 140 of the user. The hinge mechanism 125 is thus positioned where the hinge axis 130 is aligned with the rotational axis of the knee 110 that allows flexion and extension of the lower leg 140.

In the version of FIG. 1, the first frame 115 includes a first rigid member 145 and the second frame 120 includes a second rigid member 150. The first rigid member 145 is sized and shaped to fit on a body part of the user, such as the upper leg 135. The first rigid member 145 includes a transverse arch 155 that connects two longitudinally extending portions 160 (only one shown in FIG. 1). The transverse arch 155 is positionable on the front of the upper leg 135, and the longitudinally extending portions 160 extend along the side of the upper leg 135 generally in a line that intersects the axis 130 of the hinging mechanism 125. The second rigid member 150 is sized and shaped to fit on a body part opposite the joint of interest, such as a lower leg 140 in the version of FIG. 1. The second rigid member includes a transverse arch 165 that connects two longitudinally extending portions 170 (only one shown in FIG. 1). The transverse arch 165 is positionable on the back of the lower leg 140 or calf, and the longitudinally extending portions 170 extend from the transverse arch 165 of the second rigid member 150 along the side of the lower leg 140 toward the knee 110 generally in line that intersects the axis 130 of the hinging mechanism 125. The sizes and shapes of the first rigid member 145 and the second rigid member 150 can be modified to fit other body parts so other joints can be stabilized. The first rigid member 145 and the second rigid member 150 can also be further modified. For example, the transverse arch 155 of the first frame 115 can be reversed so that it fits on the back of the upper leg 135 and/or the transverse arch 155 can extend further around the circumference of the upper leg 135. Similarly, the transverse arch 165 of the second frame 120 can be reversed so that it fits on the front of the lower leg 140 and/or the transverse arch 165 can extend further around the circumference of the lower leg 140. The first rigid member 145 and the second rigid member 150 are made of a material selected to be lightweight to and maintain rigidity under normal operating conditions of the brace. For example, the first rigid member 145 and the second rigid member 150 can be made of one or more of any known materials useful for braces of this type, such as aluminum and/or an aluminum alloy.

The first frame 115 is securable on the first body part, such as the upper leg 135, by a first frame connection system 175. The first frame connection system 175 includes one or more flexible straps 180, each having a hook 185 at both ends of the flexible strap 180. Each hook 185 is attachable to one or more protrusions 190 located on both sides of the first frame 115 (only one side shown in FIG. 1). For example, a protrusion 190 may be provided on or near the transverse arch 155 of the first rigid member 145 and another protrusion may be provided at a similar location on the other side of the first rigid member 145. A flexible strap 180 may be installed by connecting the hooks 185 on both ends of the flexible strap 180 to the respective protrusions 190 so that the flexible strap 180 extends about the user's body part on the opposite side of the transverse arch 155. The flexible strap 180 can then be tightened, such as by being elastic and/or by being adjustable in length, such as by having a portion that folds over itself and attaches to the flexible strap 180 by Velcro or the like. Another set of protrusions 190 are provided on the longitudinally extending portions 160, and another strap 180 with hooks 185 is provided for securing the longitudinally extending portions to the user's body part in similar manner and as shown in FIG. 1. Any other securing system can be utilized.

The second frame 120 is securable on the second body part, such as the lower leg 140, by a second frame connection system 195. The second frame connection system 195 includes one or more flexible straps 180, each having a hook 185 at both ends of the flexible strap 180. Each hook 185 is attachable to one or more protrusions 200 located on both sides of the second frame 120 (only one side shown in FIG. 1). For example, a protrusion 200 may be provided on or near the transverse arch 165 of the second rigid member 150 and another protrusion may be provided at a similar location on the other side of the second rigid member 150. A flexible strap 180 may be installed by connecting the hooks 185 on both ends of the flexible strap 180 to the respective protrusions 200 so that the flexible strap 180 extends about the user's body part on the opposite side of the transverse arch 165. The flexible strap 180 can then be tightened, such as by being elastic and/or by being adjustable in length, such as by having a portion that folds over itself and attaches to the flexible strap 180 by Velcro or the like. Another set of protrusions 200 are provided on the longitudinally extending portions 170, and another strap 180 with hooks 185 is provided for securing the longitudinally extending portions to the user's body part in similar manner and as shown in FIG. 1. The set of protrusions 200 on the longitudinally extending portion may be additionally secured with another strap 180 that extends around the body part in the opposite direction as the other strap 180, as shown in FIG. 1. Any other securing system can be utilized.

The joint brace 100 of the present invention also includes a biasing system 205. The biasing system 205 biases the first frame 115 relative to the hinge mechanism 125 and/or the second frame 120 towards an extended position. Thus, when secured onto a user, the user's joint is biased in an extended position. In the version of FIG. 1, the biasing system 205 urges the first frame 215 into the position it would be in when the user's leg is straight. Accordingly, as the joint is flexed and the first frame 115 rotates toward the second frame 120, the biasing system 205 creates a bias that applies a force against the first frame 115 to urge it back to the fully extended or straight position. The biasing system 205 assists the user as the user tries to stand or otherwise straighten his or her leg or other body part.

In the version of FIG. 1, the biasing system 205 comprises a compression spring 210. When compressed, the compression spring 210 offers a resistance force. The compression spring is attached to the first frame 115 at a location along the midline so that when the first frame 115 is secured on a user's body part, the compression spring 210 is positioned generally on the midline of the body part. Alternatively, the compression spring 210 can be connected at a position off the midline in the direction of flexion of the joint. The other end of the compression spring 210 is connected to the hinge mechanism 125 at any position where a bias will be generated during flexion. For example, a connector 215 can be provided that is offset from the axis 130 of the hinge mechanism 125 on the side where flexion of the joint will occur. Alternatively, the connector 215 can be positioned at the axis 130 of the hinge mechanism 125 if the other end of the compression spring 210 is attached to the first frame 115 in a position where flexion of the joint will cause compression of the compression spring 215. In another version, the compression spring 215 can be connected to the second frame 120 instead of to the hinge mechanism 125. The biasing system 205 may include a second compression spring 215 attached to the other side of the joint brace (not shown) so that bias is applied to both sides of the joint. This provides additional stability and less twisting of the joint.

In one particular version, as shown in FIG. 1, the compression spring 210 includes a loop 220 that fits around a projection 190 on the first frame 115. The loop 220 can rotate on the projection 190. As the first frame 115 moves when the joint is flexed, the distance between the projection 190 and the connector 215 is shortened, and the compression spring 215 is compressed and generates a force that urges the first frame 115 back to the extended position.

The joint brace 100 having the bias system 205 offers significant advantages over existing joint braces. The joint brace 100 relieves weight on an injured joint. In addition, the joint brace 100, when used as a knee brace, can help a user stand from a crouch or from a seated position. The joint brace 100 can also be used to support or enhance athletic performance. For example, in track and field, the joint brace 100 can help propel the runner at the starting blocks. The joint brace 100 can support coming to a complete stop for gymnasts landing from the vault, a balance beam, uneven bars, still rings and horizontal bar. The same landing time support can apply to jumping off ladders, parkour, cheerleading, and the like.

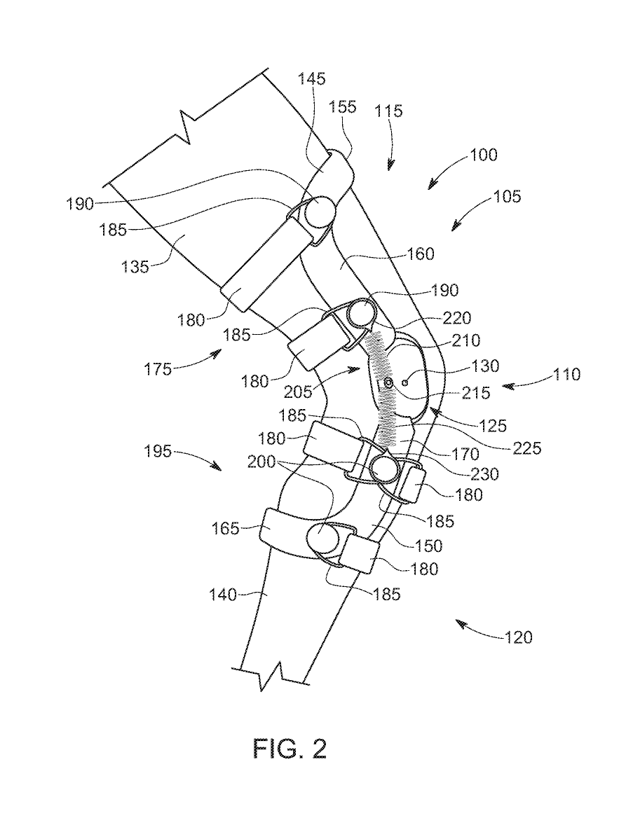

Another version of a joint brace 100 of the invention is shown in FIG. 2. In this version, the biasing system 205 comprises an additional compression spring 225 connected to the second frame 120. The additional compression spring 225 is connected to the second frame 120 at a position where the second frame is biased toward the extended position by the additional compression spring 225. In one version, the additional compression spring 225 can have a loop 230 similar to the loop 220 of the other compression spring 210 so the additional compression spring 225 can be connected to a protrusion 200 of the second frame 120.

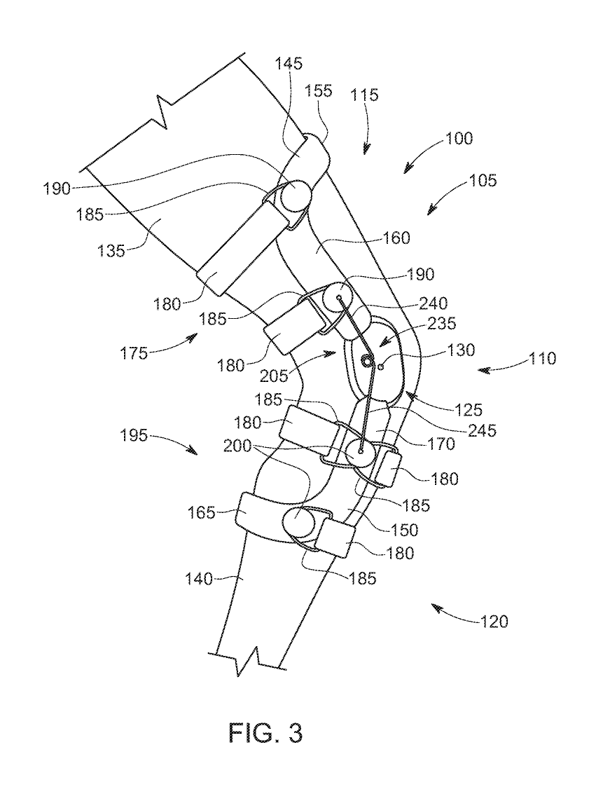

Another version of a joint brace 100 of the invention is shown in FIG. 3. In this version, the biasing system 205 comprises a torsion spring 235. A first end 240 of the torsion spring 235 is connected to the first frame 115 and a second end 245 of the torsion spring 235 is connected to the second frame 120. The torsion spring 235 is biased towards its extended or straight position. Therefore, when the first end 240 and the second end 245 are rotated toward each other, as would happen when the joint is flexed, the torsion spring 235 urges the joint back to the extended position. In other words, to flex the joint, the user would have to overcome the bias of the torsion spring 235.

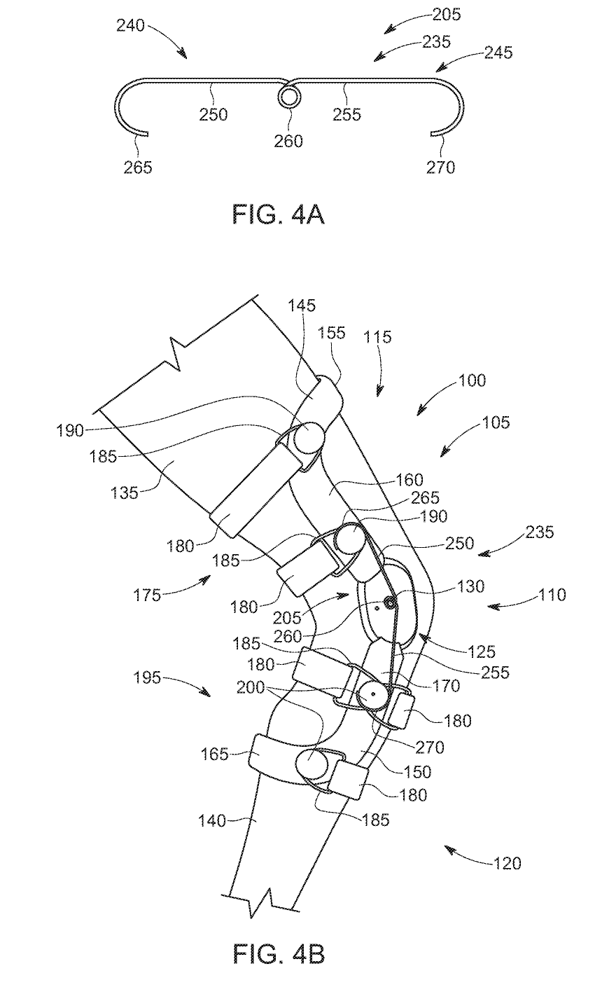

An example of a torsion spring 235 that may be used in the biasing system 205 of the invention is shown in FIG. 4A. The torsion spring 235, also know as a U-shaped or a V-shaped spring, is a spring that works by torsion or twisting. It is a flexible object that stores mechanical energy when it is twisted. When it is twisted, it exerts a torsional force in the opposite direction. The torsional force is proportional to the amount of twist. In the version of FIG. 4A, the torsion spring 235 is made up of a wire made of one or more of stainless steel, eligloy, Iconel 600, 718, x75, polyetherimide, polyurethane, beryllium copper, Hastelloy, phosphor bronze, chrome silicon, chrome alloy, high-carbon steel music wire, hard-drawn MB carbon steel, chrome vanadium, brass, spring steel, carbon spring steel, C1075 steel, H11/H13 steel, alloy steel, AISI C1075, AISI 6150, and any other known metal or alloy, such as those described in the ASM Handbook from ASM International which is incorporated herein by reference in its entirety. The metals can be hard drawn, passivated, electroplated, galvanized, and/or finished with gold irridite. The wire has a straight first end or leg 250, a straight second end or leg 255, and a coil portion 260 between the first leg 250 and the second leg 255. As the first leg 250 and the second leg 255 rotate toward each other about the coil portion 260, the coil portion 260 generates the opposing torsional force due to the elastic properties of the wire.

In one particular version, the torsion spring 235 is easily connectable and disconnectable to the joint brace 100. The torsion spring 235 can be equipped with connectors on each of its ends. For example, the first leg 250 of the torsion spring 235 can terminate in a first hook portion 265, and the second leg 255 of the torsion spring 235 can terminate in a second hook portion 270. As can be seen in FIG. 4B, the first hook portion 265 can be easily hooked onto a protrusion on the first frame 115, and the second hook portion 270 can be easily hooked onto a protrusion 200 on the second frame 120. The coil portion 260 can be positioned in proximity to the axis 130 of the hinge mechanism 125 or it can be offset therefrom. The coil portion 260 can be attached to the hinge mechanism 125 or it can be free floating. As is apparent from FIG. 4B, as the joint is flexed, the torsion spring 235 is rotated and a torsional force is generated that urges the joint back to its extended position.

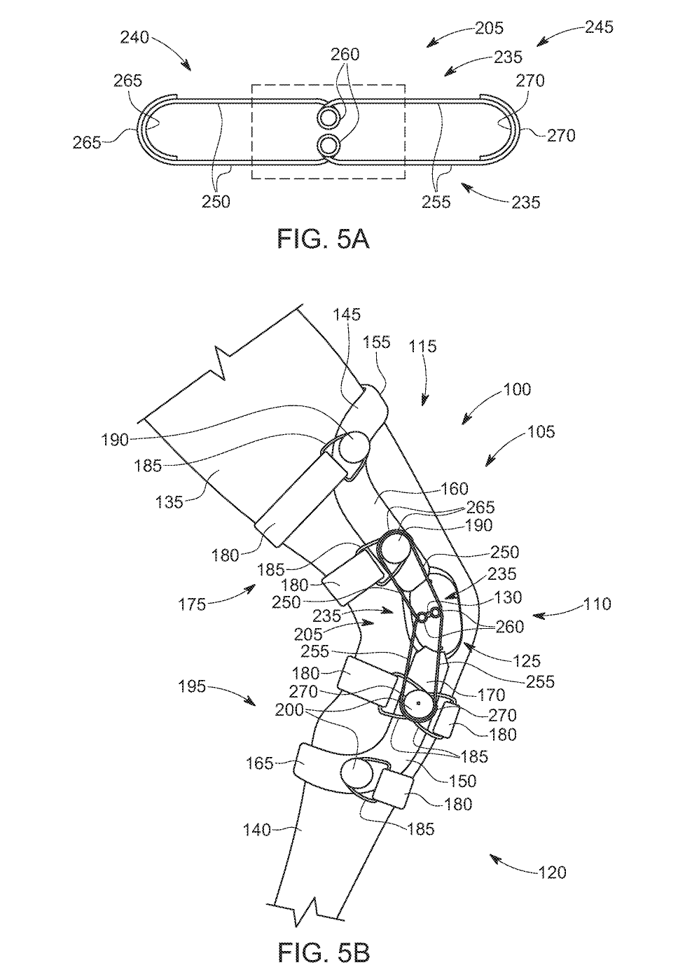

Another version of a biasing system 205 is shown in FIG. 5A. In this version, the biasing system 205 comprises a first torsion spring 235 and a second torsion spring 235 that is the same as but is positioned so that it faces the first torsion spring 235. A housing 275 can optionally be provided to contain the opposing torsion springs 235. The opposing torsion springs 235 arrangement is shown installed in FIG. 5B. This version offers advantages. For example, because there are two torsion springs 235, the bias force is doubled and additional stability is provided to the joint.

The spring rate or spring constant and the resulting amount of torsion bias applied depends upon chosen material, inner and outer spring diameters and total number of coils. In one version, the torsion spring 235 or combination of dual torsion springs 235 generates a spring load or rate of from about 0.001 lb/degree to about 500 lb/degree, more preferably from about 0.01 lb/degree to about 100 lb/degree, and most preferably from about 0.1 lb/degree to about 10.0 lb/degree. In one particular version, using music wire with spring wire diameter of about 0.150 in, an outer coil 260 diameter of about 1.1 in, an inner coil 260 diameter of about 0.8 in, torsion leg 250, 255 lengths of about 5 in, about 9 active coils in the coil 260, and a spring index of 6.33 produces a torque of up to about 54.1 in-lbs. For a person that weighs about 150 pounds, this amount of torque offers significant reduction in the torque felt by the knee.

The biasing system 205 can be permanently attached to the joint brace 100 or can be connectable and disconnectable thereto. The connectable version offers some additional advantages. For example, the biasing system 205 can be installed at selective times. There may be certain activities when it would be desirable to have the biasing system 205 installed and some activities when it would be better to have the biasing system 205 uninstalled. Also, there may be stages during the rehabilitation of an injured joint when it is desirable to have the biasing system 205 and stages when it is less desirable. In one version of the invention, a kit made up of multiple biasing systems 205 can be provided, and each biasing system 205 can provide a different biasing force. A user can install a biasing system 205 of a predetermined strength depending on the activity and/or stage of recovery.

In one version, a kit of torsion spring 235 can be provided that includes a plurality of torsion springs 235. The kit may include two or more torsion springs 235 that each have a different torque associated with it. For example, one torsion spring 235 has a spring load or rate of from about 0.5 lb/degree to about 9.0 lb/degree, more preferably from about 1 to about 5 lb/degree, and the second torsion spring 235 has a spring load or rate from about 3.0 lb/degree to about 10 lb/degree, more preferably from about 5 lb/degree to about 8.5 lb/degree. The kit may alternatively contain a two or more pairs of torsion springs 235, each pair being the same and each being different from another pair.

The bias system 205 can be designed to operate across any desired range of flexion/extension movement of the joint. For example, in one version, the bias system 205 applies the bias toward the extension position for at least the first 30 degrees of flexion from full extension. In another version, the bias system 205 applies the bias toward the extension position for at least the first 60 degrees of flexion from full extension. In another version, the bias system 205 applies the bias toward the extension position for at least the first 90 degrees of flexion from full extension. In another version, the bias system 205 applies the bias toward the extension position for at least the first 120 degrees of flexion from full extension. In another version, the bias system 205 applies the bias toward the extension position for at least the first 150 degrees of flexion from full extension.

The compression and/or torsion springs of the bias system 205 can utilize any end termination, including but not limited to closed and square end type, closed and ground end type, double closed type and/or open end type. Each end type allows the spring to behave uniquely. In one version, an open end type is used. The open end type delivers a compression spring having less force and more elasticity to all active coils. In another version, a double closed end type is used, and it provides more stability to springs that are very long but have a narrow outside diameter. In addition, the exterior spring shape can be modified to provide differing characteristics. For example, a compression spring can be shaped like a rod, a barrel, a cone, or a magazine. In one version, a conical spring is used to produce a non-linear force that does not exert a constant amount of force when compressed as may be desired in some cases. In another version, a barrel spring is used, and it produces a non-linear force and also provides stability to the spring due to the concave or convex shape while reducing the compressed solid height.

Though the bias system 205 has been described by examples using one or more compression springs and/or one or more torsion springs, other types of biasing mechanisms can be employed. For example, the bias system 205 can comprise one or more of conical springs, extension springs, wave springs, wire forms, Belleville washers, constant force specialty springs, and stamped metal forms, and any other similar type of springs.

The joint brace 100 and the biasing system 205 herein disclosed and described provides a solution to the shortcomings in the prior art through the provision of a device and method for enhancing the dampening or spring capacity of the joint brace 100 by use of a biasing system 205 which may be in the form of a retrofit kit or may be permanently installed. The knee brace 100 improves joint protection during active recovery. For example, an athlete recovering from a sprained knee who is still active and wearing an knee brace may install the biasing system 205 to provide added support to the dampening ability of the joint brace.

Although the present invention has been described in considerable detail with regard to certain preferred versions thereof, other versions are possible, and alterations, permutations and equivalents of the version shown will become apparent to those skilled in the art upon a reading of the specification and study of the drawings. For example, the cooperating components may be reversed or provided in additional or fewer number. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

What is claimed is:

1. A joint brace comprising:
a first frame adapted to be secured onto a body part on the first side of a joint,
a second frame adapted to be secured onto a body part on the other side of a joint,
a hinge mechanism connecting the first frame to the second frame so the first frame and the second frame can rotate relative to one another about a hinge axis that is adapted to be generally aligned with the axis of rotation of the joint, and
a bias system that biases the first frame away from the second frame and towards an extended position, wherein the bias is applied for more than about 30 degrees of flexion of the first frame relative to the second frame.

2. A joint brace according to claim 1 wherein the bias system comprises a torsion spring.

3. A joint brace according to claim 2 wherein the bias system has a spring rate of from about 0.001 lb/degree to about 500 lb/degree.

4. A joint brace according to claim 2 wherein the bias system has a spring rate of from about 0.01 lb/degree to about 100 lb/degree.

5. A joint brace according to claim 2 wherein the bias system has a spring rate of from about 0.1 lb/degree to about 10.0 lb/degree.

6. A joint brace according to claim 1 wherein the bias system is connectable to a protrusion on the first frame and a protrusion on the second frame.

7. A joint brace according to claim 1 wherein the bias system is removably connectable to the brace so the bias system can be selectively installed and removable.

8. A joint brace according to claim 1 wherein the bias system comprises two torsion springs that face one another.

9. A joint brace comprising:
a first frame adapted to be secured onto a body part on the first side of a joint,
a second frame adapted to be secured onto a body part on the other side of a joint,
a hinge mechanism connecting the first frame to the second frame so the first frame and the second frame can rotate relative to one another about a hinge axis that is adapted to be generally aligned with the axis of rotation of the joint, and
a bias system that biases the first frame away from the second frame and towards an extended position, wherein the bias system is removable so that it can be selectively installed and removed during a treatment period of a user.

10. A joint brace according to claim 9 wherein the bias system is removably attachable to a protrusion on the first frame and a protrusion on the second frame.

11. A joint brace according to claim 9 wherein the bias system can be selectively removed from the joint brace and reinstalled onto the joint brace without removing the joint brace from the joint.

12. A joint brace according to claim 9 wherein the bias system comprises a plurality of torsion springs.

13. A joint brace according to claim 12 wherein the plurality of torsion springs are connected to the joint brace at the same time.

14. A joint brace according to claim 9 wherein the bias system comprises a plurality of springs, and wherein the plurality of springs each have a different spring rate, a different end type, or a different spring shape and are connectable to the joint brace at different times to adjust the amount of bias of the biasing system.

15. A method of bracing a joint, the method comprising:
securing a first frame to a body part on a first side of a joint;
securing a second frame to a body part on the other side of the joint, the first and second frames being connected to one another by a hinge mechanism;
attaching to at least the first frame a biasing system that biases the first frame toward an extended position; and
removing the biasing system from the first frame when it is desired to remove the bias.

16. A method according to claim 15 further comprising reattaching the biasing system to the first frame when it is desire to have the bias.

17. A method according to claim 15 comprising attaching the biasing system to both the first frame and the second frame.

18. A method according to claim 15 wherein the biasing system comprises a plurality of springs, wherein the step of removing the biasing system comprises removing a first spring, and further comprising the step of attaching a second spring to the first frame, the second spring having a different spring rate, a different end type, or a different spring shape than the first spring.
